(19) United States
(12) Patent Application Publication
(10) Pub. No.: US 2012/0203105 A1
(43) Pub. Date: Aug. 9, 2012

(54) ULTRASOUND DIAGNOSTIC APPARATUS AND ULTRASOUND IMAGE PRODUCING METHOD

(75) Inventors: Katsuya YAMAMOTO, Kanagawa (JP); Yuji OHSHIMA, Kanagawa (JP)

(73) Assignee: FUJIFILM CORPORATION, Tokyo (JP)

(21) Appl. No.: 13/335,468

(22) Filed: Dec. 22, 2011

(30) Foreign Application Priority Data
Feb. 9, 2011 (JP) 2011-025882

(51) Int. Cl. A61B 8/14

(52) U.S. Cl. 600/443

(57) ABSTRACT

An ultrasound diagnostic apparatus comprises an ultrasound probe including a transducer array, a transmitter for transmitting an ultrasonic beam from the transducer array toward a subject, an image producer for producing an ultrasound image based on a reception signal outputted from the transducer array having received ultrasonic echoes from the subject, a temperature sensor for detecting an internal temperature of the ultrasound probe, a channel selector for selecting simultaneously available channels for reception from a plurality of channels of the ultrasound probe, and a controller for controlling the channel selector so as to reduce a number of selected simultaneously available channels for reception as the internal temperature of the ultrasound probe detected by the temperature sensor increases and as a measuring depth decreases.

### BACKGROUND OF THE INVENTION

The present invention relates to an ultrasound diagnostic apparatus and an ultrasound image producing method and particularly to reduction of the amount of heat generated in an ultrasound probe of an ultrasound diagnostic apparatus for giving a diagnosis based on an ultrasound image produced by transmission and reception of ultrasonic waves from a transducer array of the ultrasound probe.

Conventionally, ultrasound diagnostic apparatus using ultrasound images are employed in the medical field. In general, this type of ultrasound diagnostic apparatus comprises an ultrasound probe having a built-in transducer array and an apparatus body connected to the ultrasound probe. The ultrasound probe transmits ultrasonic waves toward a subject, receives ultrasonic echoes from the subject, and the apparatus body electrically processes the reception signals to generate an ultrasound image.

With such ultrasound diagnostic apparatus, heat is generated in the transducer array as it transmits ultrasonic waves.

The ultrasound probe is often encased in a housing of a size that can be readily held by an operator in a single hand because normally a diagnosis is given as the operator places the ultrasound transmission/reception surface of the transducer array in contact with a subject's surface by holding the ultrasound probe in a single hand. Therefore, the heat generated in the transducer array may raise the temperature inside the housing of the ultrasound probe.

In recent years, there has been proposed an ultrasound diagnostic apparatus having an ultrasound probe with a built-in circuit board for signal processing for effecting digital processing of a reception signal outputted from the transducer array before transmitting the reception signal to the apparatus body via wireless or wired communication in order to reduce the effects of noise and obtain a high-quality ultrasound image.

The ultrasound probe that effects digital processing of this kind is subject to generation of heat in the circuit board also during processing of the reception signals, and therefore the temperature rise in the housing needs to be suppressed to assure stable operations of the circuits on the board.

As for a countermeasure against the temperature rise in the ultrasound probe, reference is made to JP 2005-253776 A describing an ultrasound diagnostic apparatus wherein the conditions for actuating the transducer array are automatically changed according to the temperature of the surface of the ultrasound probe. The temperature of the surface of the ultrasound probe is kept at an appropriate temperature by reducing, for example, the actuating voltage, number of apertures for transmission, repetition frequency of the transmission pulse, and the frame rate as the surface temperature increases.

### SUMMARY OF THE INVENTION

However, the apparatus described in JP 2005-253776 A where the conditions for actuating the transducer array for transmission are changed cannot cope with the heat generated by the reception process in the ultrasound probe performing the above digital processing.

An object of the present invention is to eliminate the above problems associated with the prior art and provide an ultrasound diagnostic apparatus and an ultrasound image producing method enabling acquisition of a high-quality ultrasound image while suppressing the temperature rise inside the ultrasound probe.

An ultrasound diagnostic apparatus according to the present invention comprises:

an ultrasound probe including a transducer array;

a transmitter for transmitting an ultrasonic beam from the transducer array toward a subject;

an image producer for producing an ultrasound image based on a reception signal outputted from the transducer array having received ultrasonic echoes from the subject;

a temperature sensor for detecting an internal temperature of the ultrasound probe;

a channel selector for selecting simultaneously available channels for reception from a plurality of channels of the ultrasound probe; and

a controller for controlling the channel selector so as to reduce a number of selected simultaneously available channels for reception as the internal temperature of the ultrasound probe detected by the temperature sensor increases and as a measuring depth decreases.

A method of producing an ultrasound image according to the present invention comprises the steps of:

detecting an internal temperature of an ultrasound probe including a transducer array;

selecting simultaneously available channels for reception from a plurality of channels of the ultrasound probe so as to reduce a number of selected simultaneously available channels for reception as the detected internal temperature of the ultrasound probe increases and as a measuring depth decreases; and

transmitting an ultrasonic beam from the transducer array toward a subject and producing an ultrasound image based on a reception signal outputted from the transducer array having received ultrasonic echoes from the subject.

### BRIEF DESCRIPTION OF DRAWINGS

FIG. 1 is a block diagram illustrating a configuration of an ultrasound diagnostic apparatus according to Embodiment 1 of the invention.

FIG. 2 illustrates an imaging region divided into three regions according to a measuring depth.

FIG. 3 is a graph illustrating a temporal variation in temperature inside an ultrasound probe and temperature thresholds according to Embodiment 1.

FIG. 4 illustrates available channels and non-available channels selected in Embodiment 1.

FIG. 5 illustrates available channels and non-available channels selected in Embodiment 2.

### DETAILED DESCRIPTION OF THE INVENTION

Embodiments of the present invention will be described below based on the appended drawings.

### Embodiment 1

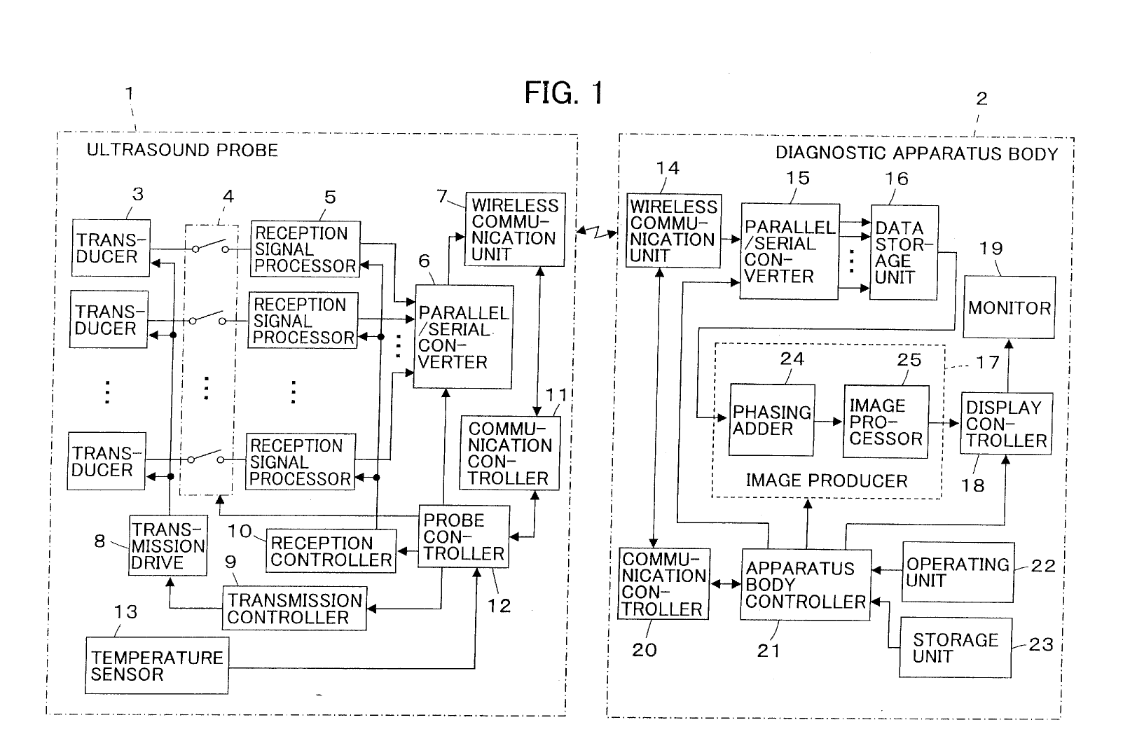

FIG. 1 illustrates a configuration of an ultrasound diagnostic apparatus according to Embodiment 1 of the invention. The ultrasound diagnostic apparatus comprises an ultrasound probe 1 and a diagnostic apparatus body 2 that is connected to the ultrasound probe 1 via wireless communication.

The ultrasound probe 1 comprises a plurality of ultrasound transducers 3 constituting a plurality of channels of a unidimensional or two-dimensional transducer array, and the transducers 3 are connected via a channel selector 4 to reception signal processors 5, which in turn are connected to a wireless communication unit 7 via a parallel/serial converter 6. The transducers 3 are connected to a transmission controller 9 via a transmission drive 8, and the reception signal processors 5 are connected to a reception controller 10, while the wireless communication unit 7 is connected to a communication controller 11. The channel selector 4, the parallel/serial converter 6, the transmission controller 9, the reception controller 10, and the communication controller 11 are connected to a probe controller 12. The ultrasound probe 1 has a built-in temperature sensor 13 for detecting the temperature inside the ultrasound probe 1, and the temperature sensor 13 is connected to the probe controller 12.

The temperature sensor 13 is preferably located near the reception signal processors 5, where heat is expected to develop during the operation of the ultrasound diagnostic apparatus.

The transducers 3 each transmit ultrasonic waves according to actuation signals supplied from the transmission drive 8 and receive ultrasonic echoes from the subject to output reception signals. Each of the transducers 3 is composed of an oscillator comprising a piezoelectric body and electrodes each provided on both ends of the piezoelectric body. The piezoelectric body may be composed, for example, of a piezoelectric ceramic typified by a PZT (titanate zirconate lead), a polymeric piezoelectric device typified by PVDF (polyvinylidene flouride), and a monocrystal typified by PMN-PT (lead magnesium niobate lead titanate solid solution).

When the electrodes of each of the oscillators are supplied with a pulsed voltage or a continuous-wave voltage, the piezoelectric body expands and contracts to cause the oscillator to produce pulsed or continuous ultrasonic waves. These ultrasonic waves are combined to form an ultrasonic beam. Upon reception of propagating ultrasonic waves, each oscillator expands and contracts to produce an electric signal, which is then outputted as an ultrasonic reception signal.

The transmission drive 8 includes, for example, a plurality of pulsers and adjusts the delay amounts of actuation signals for the respective transducers 3 based on a transmission delay pattern selected by the transmission controller 9 so that the ultrasonic waves transmitted from the transducers 3 form a broad ultrasonic beam covering an area of a tissue of the subject and supplies the transducers 3 with adjusted actuation signals.

The channel selector 4 comprises a plurality of switches connecting and disconnecting the transducers 3 and the corresponding reception signal processors 5 and selects the simultaneously available channels for reception among the channels of the transducer array according to an instruction from the probe controller 12 to connect the transducers 3 of the selected channels to the corresponding reception signal processors 5.

Under the control of the reception controller 10, the individual channels of the reception signal processors 4 allow the reception signal outputted from the corresponding transducers 3 to undergo quadrature detection or quadrature sampling process to produce a complex base band signal, samples the complex base band signals to generate sample data containing information on the area of the tissue, and supplies the parallel/serial converter 6 with the sample data. The reception signal processors 5 may generate sample data by performing high-efficiency coding data compression on the data obtained by sampling the complex baseband signals.

The parallel/serial converter 6 converts the parallel sample data generated by the reception signal processors 5 having a plurality of channels into serial sample data.

The wireless communication section 7 performs carrier modulation based on the serial sample data to generate transmission signals and supplies an antenna with the transmission signals so that the antenna transmits radio waves to transmit serial sample data. The modulation methods that may be employed herein include ASK (Amplitude Shift Keying), PSK (Phase Shift Keying), QPSK (Quadrature Phase Shift Keying), and 16QAM (16 Quadrature Amplitude Modulation).

The wireless communication unit 7 transmits the sample data to the diagnostic apparatus body 2 through wireless communication with the diagnostic apparatus body 2, receives various control signals from the diagnostic apparatus body 2, and outputs the received control signals to the communication controller 11. The communication controller 11 controls the wireless communication unit 7 so that the sample data is transmitted with a transmission wave intensity that is set by the probe controller 12 and outputs various control signals received by the wireless communication unit 7 to the probe controller 12.

The temperature sensor 13 detects and outputs an internal temperature of the ultrasound probe 1 to the probe controller 12.

The probe controller 12 controls various components of the ultrasound probe 1 according to control signals transmitted from the diagnostic apparatus body 2. The probe controller 12 controls the ON/OFF operation of the switches of the channel selector 4 for reception according to the internal temperature T of the ultrasound probe 1 detected by the temperature sensor 13 and the measuring depth.

The ultrasound probe 1 has a built-in battery, not shown, which supplies electric power to the circuits inside the ultrasound probe 1.

The ultrasound probe 1 may be an external type probe such as linear scan type, convex scan type, and sector scan type or a probe of, for example, a radial scan type used for an ultrasound endoscope. A plurality of transducers 3 may be connected to a single multiplexer to switch the available channels for transmission.

On the other hand, the diagnostic apparatus body 2 comprises a wireless communication unit 14, which is connected to a data storage unit 16 via a serial/parallel converter 15. The data storage unit 16 is connected to an image producer 17. The image producer 17 is connected to a monitor 19 via a display controller 18. The wireless communication unit 14 is also connected to a communication controller 20; the serial/parallel converter 15, the image producer 17, the display controller 18, and the communication controller 20 are connected to an apparatus controller 21. The apparatus controller 21 is connected to an operating unit 22 for an operator to perform input operations and to a storage unit 23 for storing operation programs.

The wireless communication unit 14 transmits various control signals to the ultrasound probe 1 through wireless communication with the ultrasound probe 1. The wireless communication section 14 demodulates the signal received by the antenna to output serial sample data.

The communication controller 20 controls the wireless communication unit 14 so that various control signals are transmitted with a transmission radio wave intensity that is set by the apparatus body controller 21.

The serial/parallel converter 15 converts the serial sample data outputted from the wireless communication unit 14 into parallel sample data. The data storage unit 16 is configured by a memory, a hard disk, or the like and stores at least one frame of sample data converted by the serial/parallel converter 15.

The image producer 17 performs reception focusing on each frame of sample data read out from the data storage unit 16 to generate an image signal representing an ultrasound diagnostic image. The image producer 17 includes a phasing adder 24 and an image processor 25.

The phasing adder 24 selects one reception delay pattern from a plurality of previously stored reception delay patterns according to the reception direction set by the apparatus controller 21 and, based on the selected reception delay pattern, provides the complex baseband signals represented by the sample data with respective delays and adds them up to perform the reception focusing. This reception focusing yields a baseband signal (sound ray signal) where the ultrasonic echoes are well focused.

The image processor 25 generates a B-mode image signal, which is tomographic image information on a tissue inside the subject, according to the sound ray signal generated by the phasing adder 24. The image processor 25 includes an STC (sensitivity time control) section and a DSC (digital scan converter). The STC section corrects the sound ray signal for the attenuation due to distance according to the depth of the reflection position of the ultrasonic waves. The DSC converts the sound ray signal corrected by the STC into an image signal compatible with the scanning method of an ordinary television signal (raster conversion), and generates a B mode image signal through required image processing such as contrast processing.

The display controller 18 causes the monitor 19 to display an ultrasound diagnostic image according to the image signals generated by the image producer 17. The monitor 19 includes a display device such as an LCD, for example, and displays an ultrasound diagnostic image under the control of the display controller 18.

While the serial/parallel converter 15, the image producer 17, the display controller 18, the communication controller 20, and the apparatus controller 21 in such diagnostic apparatus body 2 are each constituted by a CPU and an operation program for causing the CPU to perform various kinds of processing, they may be constituted by a digital circuit. The aforementioned operation program is stored in the storage unit 23. The recording medium in the storage unit 23 may be a flexible disk, MO, MT, RAM, CD-ROM, DVD-ROM or the like besides a built-in hard disk.

Now, the relationship between the internal temperature T of the ultrasound probe 1 and the measuring depth on the one hand and a number N of the simultaneously available channels according to Embodiment 1 will be described.

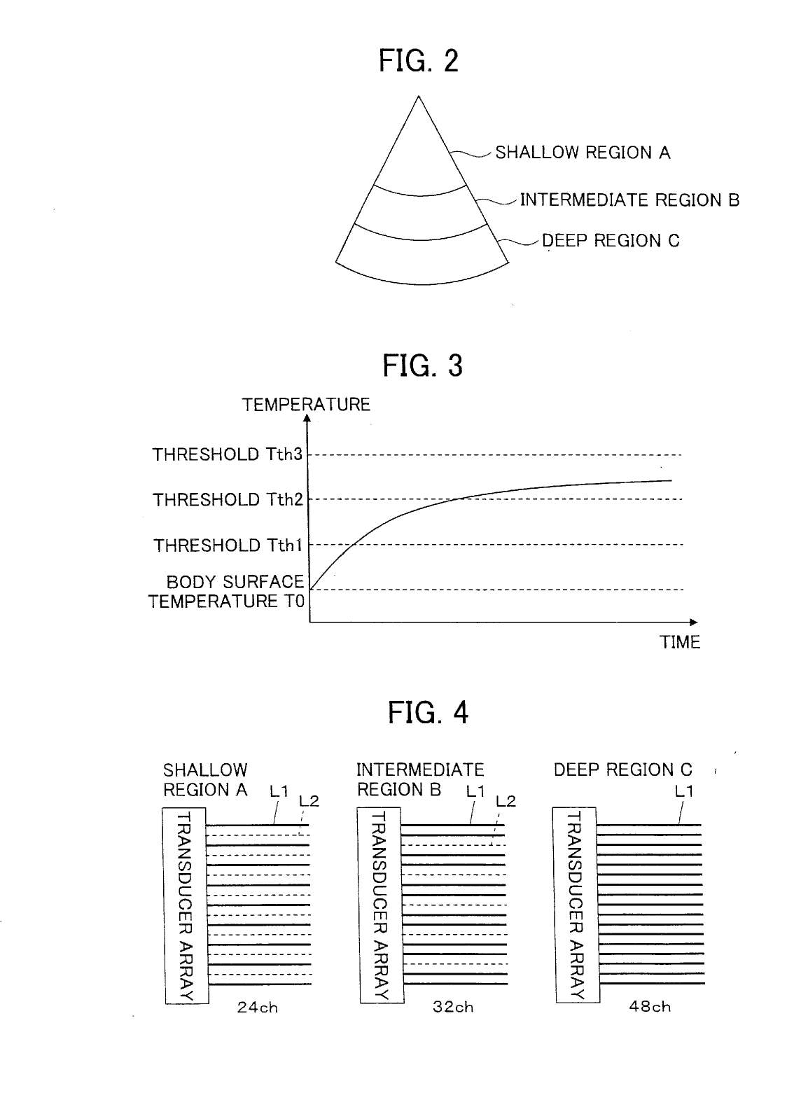

It is assumed that the imaging region is divided into three regions, a shallow region A, an intermediate region B, and a deep region C as illustrated in FIG. 2, and that three temperature thresholds, a first temperature threshold Tth1, a second temperature threshold Tth2, and a third temperature threshold Tth3, the temperature increasing from the first to the third, are previously set on the higher side of a subject's body surface temperature T0 (about 33° C.) as illustrated in FIG. 3. The first temperature threshold Tth1, the second temperature threshold Tth2, and the third temperature threshold Tth3 are set to, for example, 37° C., 40° C., and 43° C., respectively.

The number N of the simultaneously available channels for reception among the number of all the channels of the transducer array is set stepwise so as to be reduced as the internal temperature T of the ultrasound probe 1 increases and reduced as the measuring depth decreases. When, for example, the transducer array has all the 48 channels, the number N of simultaneously available channels for reception is each set to a value as shown in Table 1 depending on the internal temperature T of the ultrasound probe 1 and the measuring depth.

TABLE 1

| | T0 ≦ T < Tth1 | Tth1 ≦ T < Tth2 | Tth2 ≦ T < Tth3 |
|---|---|---|---|
| Shallow region A | 24 CH | 16 CH | 8 CH |
| Intermediate | 32 CH | 24 CH | 16 CH |
| region B | | | |
| Deep region C | 48 CH | 32 CH | 24 CH |

Thus, when the internal temperature T of the ultrasound probe 1 is T0≦T<Tth1, the number N of simultaneously available channels for reception is set to 24 for the shallow region A, 32 for the intermediate region B, and 48 for the deep region. Likewise, when the internal temperature T of the ultrasound probe 1 is Tth1≦T<Tth2, the number N is set to 16 for the shallow region A, 24 for the intermediate region B, and 32 for the deep region; when the internal temperature T of the ultrasound probe 1 is Tth2≦T<Tth3, the number N is set to 8 for the shallow region A, 16 for the intermediate region B, and 24 for the deep region.

When the internal temperature T of the ultrasound probe 1 reaches or exceeds the third temperature threshold Tth3, the transmission and reception of the ultrasonic waves are terminated.

The number N of simultaneously available channels for reception for the shallow region A, the intermediate region B, and the deep region C in the individual temperature ranges may have been previously entered from the operating unit 22 of the diagnostic apparatus body 2 and may be stored in the storage unit 23 as table of the number of simultaneously available channels.

The transmission drive 8 is directly connected to the transducers 3 without the intermediary of the channel selector 4 and the transmission of the ultrasonic waves is carried out using all the channels of the transducer array.

Next, the operation of Embodiment 1 will be described.

When ultrasound diagnosis is started, the internal temperature T of the ultrasound probe 1 is first detected by the temperature sensor 13 and wirelessly transmitted to the diagnostic apparatus body 2 via the probe controller 12, the communication controller 11, and the wireless communication unit 7. The internal temperature T received by the wireless communication unit 14 of the diagnostic apparatus body 2 is inputted to the apparatus body controller 21 via the communication controller 20.

The apparatus body controller 21 reads the table of the number of simultaneously available channels stored in the storage unit 23 to set the number N of simultaneously available channels for reception for each of the shallow region A, the intermediate region B, and the deep region C according to the entered internal temperature T of the ultrasound probe 1. The number N of simultaneously available channels is wirelessly transmitted from the apparatus body controller 21 to the ultrasound probe 1 via the communication controller 20 and the wireless communication unit 14 and inputted to the probe controller 12 via the wireless communication unit 7 and the communication controller 11 of the ultrasound probe 1.

The transmission drive 8 is operated by the probe controller 12 via the transmission controller 9, and ultrasonic waves are transmitted from the transducers 3 of all the channels of the transducer array according to the actuation signals supplied from the transmission drive 8. Thus, the reception signals are outputted from the transducers 3 having received ultrasonic echoes from the subject as the probe controller 12 controls the ON/OFF operations of the individual switches of the channel selector 4 so that the number of simultaneously available channels becomes the number N set according to the measuring depth. Because, at the earlier stage of reception, the ultrasonic echoes from the shallow region A are received, the switches of the channel selector 4 corresponding to the number N of simultaneously available channels that for the shallow region A are turned ON, while the remaining switches are turned OFF. When the reception of the ultrasonic echoes from the intermediate region B is started after the reception of the ultrasonic echoes from the shallow region A, the switches of the channel selector 4 corresponding to the number N of simultaneously available channels set for the intermediate region B are turned ON, while the remaining switches are turned OFF. When the reception of the ultrasonic echoes from the deep region C is started, the switches of the channel selector 4 corresponding to the number N of simultaneously available channels set for the deep region C are turned ON, while the remaining switches are turned OFF.

When, for example, the internal temperature T of the ultrasound probe 1 lies between the surface temperature T0 (about 33° C.) and the first temperature threshold Tth1 (37° C.), the channel selector 4 makes the ON/OFF control so that an available channel L1 and a non-available channel L2 are formed alternately, securing 24 channels as the number N of simultaneously available channels among a total of 48 channels of the transducer array for the shallow region A, as illustrated in FIG. 4. For the intermediate region B, two available channels L1 and one non-available channel L2 are formed in every three channels, securing 32 channels as the number N of simultaneously available channels. For the deep region C, all the switches of the channel selector 4 are turned on to select all the channels of the transducer array as available channels L1, securing 48 channels as the number N of simultaneously available channels.

Thus, even when the number N of simultaneously available channels changes, the probe controller 12 makes the ON/OFF control of the individual switches of the channel selector 4 so as to select a required number of simultaneously available channels that are substantially evenly spaced over the whole channels of the transducer array.

The reception signals from the transducers 3 of the simultaneously available channels L1 selected by the channel selector 4 are supplied to the corresponding reception signal processors 5 to produce sample data, which undergo conversion into serial data through the parallel/serial converter 6 before being transmitted wirelessly from the wireless communication unit 7 to the diagnostic apparatus body 2. The sample data received by the wireless communication unit 14 of the diagnostic apparatus body 2 are converted into parallel data through the serial/parallel converter 15 and stored in the data storage unit 16. Further, the sample data are read out from the data storage unit 16 frame by frame, and the image producer 17 generates image signals, based on which image signals the display controller 18 causes the monitor 19 to display an ultrasound diagnostic image.

When the internal temperature T of the ultrasound probe 1 increases to a temperature equal to or above the first temperature threshold Tth1 (37° C.) and below the second temperature threshold Tth2 (40° C.), the channel selector 4 forms 16, 24, and 32 simultaneously available channels L1 for the shallow region A, the intermediate region B, and the deep region C, respectively, to produce the ultrasound diagnostic image in a similar manner. When the internal temperature T of the ultrasound probe 1 increases to a temperature equal to or above the second temperature threshold Tth2 (40° C.) and below the third temperature threshold Tth3 (43° C.), the channel selector 4 forms 8, 16, and 24 simultaneously available channels L1 for the shallow region A, the intermediate region B, and the deep region C, respectively, to produce the ultrasound diagnostic image in a similar manner.

When the internal temperature T of the ultrasound probe 1 increases to a temperature equal to or above the third temperature threshold Tth3 (43° C.), transmission and reception of the ultrasonic waves are terminated until the internal temperature T decreases to under the third temperature threshold Tth3.

As described above, the internal temperature T of the ultrasound probe 1 is detected by the temperature sensor 13, and the number N of simultaneously available channels for reception is reduced accordingly as the internal temperature T increases, so that the power consumption in the reception signal processors 5 is reduced accordingly, and the heat generated in the housing of the ultrasound probe 1 also decreases accordingly. Thus, temperature rise in the ultrasound probe 1 can be suppressed while ultrasound diagnosis is continued.

Further, because the number N of simultaneously available channels for reception is reduced accordingly as the measuring depth decreases, temperature rise in the ultrasound probe 1 can be suppressed with the decrease in image quality held to a minimum.

Still further, because a required number of simultaneously available channels are selected so as to be substantially evenly spaced over the whole channels of the transducer array irrespective of the number N of simultaneously available channels as illustrated in FIG. 4, the transmission focus can be provided in positions substantially evenly spaced over the whole range of the imaging region in its scan direction to form sound rays over the whole range of the imaging region. Thus, while reduction in the number N of simultaneously available channels may lower the image quality, an ultrasound diagnostic image having an image quality that is substantially consistent over the whole screen can be produced.

### Embodiment 2

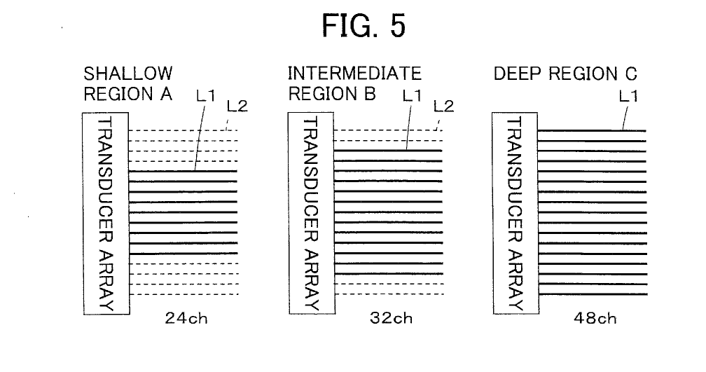

While the channel selector 4 is controlled so as to select a required number of simultaneously available channels substantially evenly spaced over the whole channels of the transducer array, the invention is not limited this way; as illustrated in FIG. 5, the channel selector 4 may be so controlled as to select a required number of channels located at the center and on both sides thereof among all the channels of the transducer array.

When, for example, the internal temperature T of the ultrasound probe 1 lies between the surface temperature T0 (about 33° C.) and the first temperature threshold Tth1 (37° C.), 24 channels located at the center are selected as available channels L1, with the remaining channels located on both sides thereof selected as non-available channels L2, among all the 48 channels of the transducer array, for the shallow region A; 32 channels located at the center are selected as available channels L1, with the remaining channels located on both sides thereof selected as non-available channels L2, among all the 48 channels of the transducer array, for the intermediate region B; and all the 48 channels of the transducer array are selected as available channels L1 for the deep region C.

When the internal temperature T of the ultrasound probe 1 increases to a temperature equal to or above the first temperature threshold Tth1 (37° C.) and below the second temperature threshold Tth2 (40° C.), 16, 24, and 32 channels are selected from the centrally located channels as available channels L1, with the remaining channels on both sides thereof selected as non-available channels, for the shallow region A, the intermediate region B, and the deep region C, respectively. Likewise, when the internal temperature T of the ultrasound probe 1 increases to a temperature equal to or above the second temperature threshold Tth2 (40° C.) and below the third temperature threshold Tth3 (43° C.), 8, 16, and 24 channels are selected from centrally located channels as available channels L1, with the remaining channels on both sides thereof selected as non-available channels, for the shallow region A, the intermediate region B, and the deep region C, respectively.

With a required number of simultaneously available channels thus selected from centrally located channels, an ultrasound diagnostic image of the central area necessary for diagnosis can be obtained without reducing the image quality even with a changing number of simultaneously available channels.

While the storage unit 23 of the diagnostic apparatus body 2 stores the table of the number of simultaneously available channels in Embodiments 1 and 2, the table of the number of simultaneously available channels may be stored in the ultrasound probe 1, so that the probe controller 12 may set the number N of simultaneously available channels for reception for each of the shallow region A, the intermediate region B, and the deep region C according to the internal temperature T of the ultrasound probe 1 detected by the temperature sensor 13.

While the ultrasound probe 1 described in Embodiments 1 and 2 comprises a transducer array having a total of 48 channels by way of example, the number of channels, 48, is only illustrative, and the present invention may likewise be applied to the ultrasound probe comprising a transducer array having another number of channels.

While the imaging region is divided into three regions, the region A, the intermediate region B, and the deep region C according to the measuring depth, and three temperature ranges, T0≦T<Tth1, Tth1≦T<Tth2, Tth2≦T<Tth3, are used for judging the internal temperature T of the ultrasound probe 1 in Embodiments 1 and 2, the present invention is not limited this way; the imaging region may be divided into two or four regions according to the measuring depth while two temperature ranges or four or more temperature ranges may be used to judge the internal temperature T of the ultrasound probe 1. In any of these cases, the number N of the simultaneously available channels for reception is so set as to be reduced stepwise as the internal temperature T of the ultrasound probe 1 increases and as the measuring depth decreases.

While the ultrasound probe 1 and the diagnostic apparatus body 2 are connected to each other by wireless communication in Embodiments 1 and 2, the invention is not limited thereto, and the ultrasound probe 1 may be connected to the diagnostic apparatus body 2 via a connection cable. Such configuration obviates the necessity to provide such components as the wireless communication unit 7 and the communication controller 11 of the ultrasound probe 1, and the wireless communication unit 14 and the communication controller 20 of the diagnostic apparatus body 2.

What is claimed is:

1. An ultrasound diagnostic apparatus comprising:
an ultrasound probe including a transducer array;
a transmitter for transmitting an ultrasonic beam from the transducer array toward a subject;
an image producer for producing an ultrasound image based on a reception signal outputted from the transducer array having received ultrasonic echoes from the subject;
a temperature sensor for detecting an internal temperature of the ultrasound probe;
a channel selector for selecting simultaneously available channels for reception from a plurality of channels of the ultrasound probe; and
a controller for controlling the channel selector so as to reduce a number of selected simultaneously available channels for reception as the internal temperature of the ultrasound probe detected by the temperature sensor increases and as a measuring depth decreases.

2. The ultrasound diagnostic apparatus according to claim 1, wherein the controller controls the channel selector to select a required number of simultaneously available channels so as to be substantially evenly spaced over the whole of the plurality of channels of the ultrasound probe.

3. The ultrasound diagnostic apparatus according to claim 1, wherein the controller controls the channel selector to select centrally located channels and other channels located on both sides of the centrally located channels from the plurality of channels of the ultrasound probe to secure a required number of simultaneously available channels.

4. The ultrasound diagnostic apparatus according to claim 1, wherein the controller controls the transmitter to transmit ultrasonic waves from all of the plurality of channels.

5. A method of producing an ultrasound image, comprising the steps of:
detecting an internal temperature of an ultrasound probe including a transducer array;
selecting simultaneously available channels for reception from a plurality of channels of the ultrasound probe so as to reduce a number of selected simultaneously available channels for reception as the detected internal temperature of the ultrasound probe increases and as a measuring depth decreases; and
transmitting an ultrasonic beam from the transducer array toward a subject and producing an ultrasound image based on a reception signal outputted from the transducer array having received ultrasonic echoes from the subject.

6. The method of producing an ultrasound image, according to claim 5, wherein a required number of simultaneously available channels substantially evenly spaced over the whole of the plurality of channels of the ultrasound probe are selected.

7. The method of producing an ultrasound image according to claim 5, wherein centrally located channels and other channels located on both sides of the centrally located channels are selected from the plurality of channels of the ultrasound probe to secure a required number of simultaneously available channels.

8. The method of producing an ultrasound image, according to claim 5, wherein ultrasonic waves are transmitted from all of the plurality of channels of the ultrasound probe.
